(19) United States
(12) Patent Application Publication
(10) Pub. No.: US 2002/0087149 A1
(43) Pub. Date: Jul. 4, 2002

(54) Ophthalmic illumination device

(76) Inventor: Brian Douglas McCary, Clayton, MO (US)

Correspondence Address: Michael L. Smith, Bausch & Lomb Inc., One Bausch & Lomb Place, Rochester, NY, 14604-2701, US

(21) Appl. No.: 09/753,420

(22) Filed: Jan. 3, 2001

(51) Int. Cl. A61B 18/18

(52) U.S. Cl. 606/4; 606/15

(57) ABSTRACT

An ophthalmic illumination device (10) includes a source of light (12) that has a spectrum primarily in a red spectrum and is substantially devoid of spectra in any other color, including a blue spectrum and a green spectrum. The red light is provided by a red light emitting diode (12) and may be used in combination with known ophthalmic illumination devices that provide a white light (31). A surgeon controls the device and can switch (36) between the red light (33) and the white light (31).

### BACKGROUND OF THE INVENTION

I. Field of the Invention

This invention relates to ophthalmic illumination devices and, more particularly, to illumination devices providing red light to a patient's eye during posterior ophthalmic surgery.

II. Description of the Related Art

Illumination during ophthalmic surgery requires a balance of competing interests between providing a surgeon with adequate light to operate and protecting a patient's retina from overexposure to potentially harmful light. Absent any harmful effects to patients' eyes, surgeons have preferred to use a bright, white light throughout their operations. However, patients' eyes are sensitive to light, and instances of retinal damage (retinal photo-toxicity) caused by lights used during posterior ophthalmic surgery have been documented. This problem has been described in detail in an article by Michels and Sternberg, “Operating Microscope-Induced Retinal Photo-toxicity; Pathophysiology, Clinical manifestations and Prevention” published in the Survey of Ophthalmology, Jan.-Feb., 1990, pages 237 to 252, which is incorporated by reference herein.

Ophthalmic surgery may generally be broken into two different classes: anterior segment surgery, and posterior segment surgery. In general, anterior segment surgery involves surgeries on the cornea or lens, or on structures that lie anterior to the lens. In contrast, posterior segment surgery involves surgeries on structures that lie behind the lens, such as the vitreous fluid and the retina. Both types of surgery are done under a surgical microscope. However, the illumination for anterior surgeries is generally supplied entirely by the operating microscope, while the illumination for posterior surgeries is generally supplied by an endoilluminator, typically through a plastic optical fiber 0.030″ in diameter. The fiber is inserted into the posterior chamber of the eye through a small slit in the sclera. Occasionally, smaller diameter fibers are used.

Light sources for ophthalmic surgery in the posterior segment traditionally provide a white light, encompassing the entire spectrum of visible colors, along with the invisible electromagnetic radiation in the infrared and ultraviolet spectra. This visible light includes red, green, and blue hues. The red hues make the red tissue of the retina visible. Some surgeons feel that the green and blue hues can help make subtle differences in tissue inside the eye more obvious. For diagnostic purposes, a white light may improve the surgeon's ability to distinguish these differences.

The initiation and extent of retinal damage due to light, photo-toxicity, depends upon the intensity, duration, and spectrum of light received by the retinal tissue. Advances in the protection against retinal damage have been based on these fundamental principles. Some ophthalmic illumination devices offer protection from particularly harmful wavelengths in the ultraviolet spectrum by the use of filters, and further protection has been suggested by devices that filter out the infrared spectrum. Protection from the intensity of the light during anterior segment surgery may be afforded by obliquely illuminating the surgical field; no such oblique illumination is available in posterior surgery. Dimmers and shutters may also be useful in protecting the retina by providing the surgeon with active control over the intensity during the course of the procedure.

There are several other effects that remain largely unexploited in the effort to minimize the risk of photo-toxicity in retinal surgery. First, it is well understood that the retina is an efficient absorber of visible light, particularly in the middle of the visible spectrum. In addition, it is generally understood that the retina is a good reflector of red light. This is the origin of the “red reflex” effect, which causes eyes to glow red in some flash photography. Further, it is well understood that exposure to light with shorter wavelengths, such as blue and green, carries a higher risk of photo-toxic injury than exposure to light with longer wavelengths. In posterior surgery, the hazard associated with wavelengths shorter than 440 nm is higher than in anterior surgery, due to the lack of absorption from the lens, according to the standards adopted by the American Conference of Governmental Industrial Hygienists (ACGIH). See 1996 TLVs and BEIs, pages 104-107.

The result is that much of the light emitted by white light sources for posterior surgery is absorbed by the patient's retina rather than being reflected back from the retina for observation by the surgeon. This results in inefficient light coupling through the endoilluminator-to-patient-to-surgeon light transmission sequence. This in turn, means that the surgeon must turn the source up to provide more light in the red regions that he can observe, at the same time increasing the intensity of the green and blue light that is less useful and more toxic. Some endoillumination sources do supply filters that filter out wavelengths shorter than the 440 nm region, but all such existing filters supply broad spectrum light of a generally white hue. Phototoxic injuries can occur during any posterior surgeries where an endoilluminator is used, including both the removal of the vitreous fluid, and direct surgery on the retina.

Thus, there is a need in the art to provide a light source for posterior surgery that is substantially red, and void of light with significant energy in the green or blue bands.

### SUMMARY OF THE INVENTION

It is in view of the above problems that the present invention was developed. The invention is a ophthalmic illumination device that produces substantially monochromatic light in the red spectrum and is counterintuitive to the current devices and practices that use bright, white light throughout ophthalmic surgery. This invention exploits the findings of research correlating phototoxicity with exposure time to short wavelength light and proposes a source of light that can be used during ophthalmic surgery.

Specifically, this invention provides an ophthalmic illumination device having a source of red light, substantially devoid of any other color, capable of reducing the risk of photoxicity.

The present invention further provides an ophthalmic illumination device having alternative selections of white light and red light and allows a surgeon the capability to change the color of light during an ophthalmic procedure.

Further features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 is a sectional view of an ophthalmic illumination device relative to a schematic illustration of an eye;

FIG. 2 is a graphic illustration of the spectral distribution for a typical source of white light;

FIG. 2A and 2B are graphic illustrations of the spectral distribution of typical ophthalmic light sources;

FIG. 2C is a graphic illustration of a spectral reflection of a typical human retina;

FIG. 3 is a schematic diagram of an alternative embodiment of the ophthalmic illumination device;

FIG. 4A, 4B, 4C, 4D, and 4E particularly illustrates combinations of optical structures that are functionally equivalent to other optical structures; and

FIG. 5 illustrates an alternative means for an ophthalmic illumination device to direct light into an eye.

### Detailed Description of the Preferred Embodiments

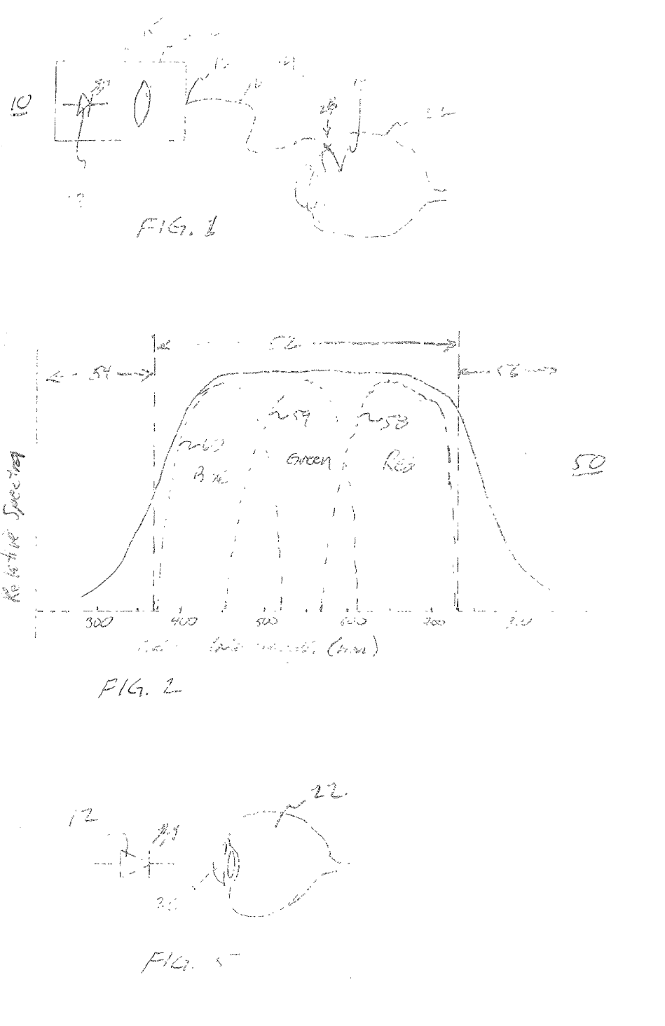

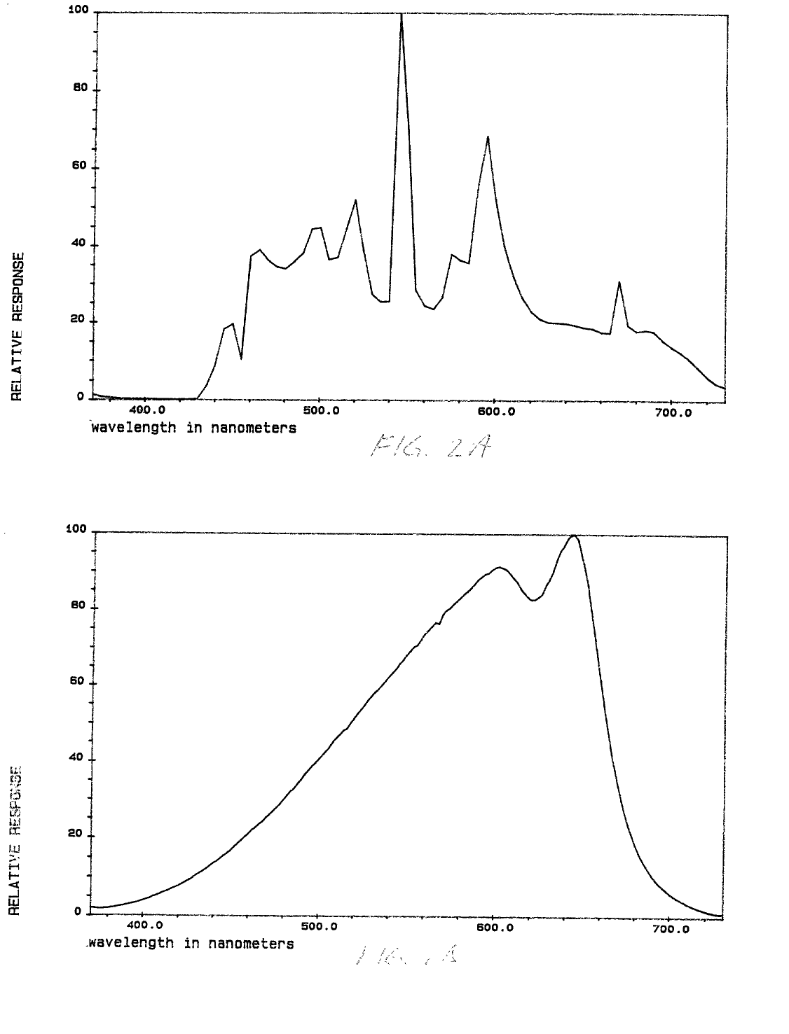

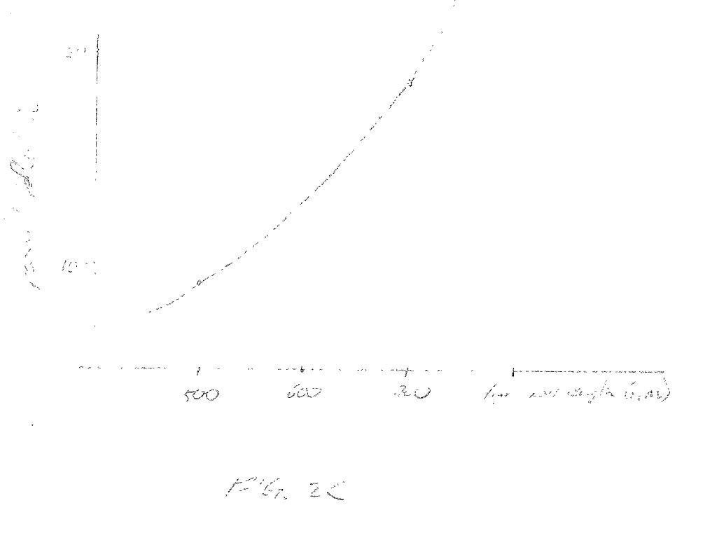

Referring to the accompanying drawings in which like reference numbers indicate like elements, FIG. 1 shows an ophthalmic illumination device 10, in accordance with the present invention. Within a housing module 15, the illumination device 10 has a red Light Emitting Diode (LED) 12 that shines a red spectrum of light 58 (see FIG. 2) through a lens 20 into the proximal end 16 of a fiber optic cable 14. The red spectrum is substantially devoid of a blue spectrum 60 and a green spectrum 59. The fiber optic cable 14 extends through the hosing module 15 and into the eye 22 at its terminal end 18 through an incision 24, thereby directing the light into the eye according to the control of a surgeon (not shown).

FIG. 2 is a graphic illustration of the spectral distribution for a typical source of visible, white light 50. The spectral energy for the white light source 50 is greatest in the visible spectrum 52, having less energy in wavelengths corresponding to the ultraviolet 54 and infrared 56 spectra. Also, shown is a graphic illustration of several spectra of color within the white light 52, including a red spectrum 58, a green spectrum 59, and a blue spectrum 60. The red spectrum 58 of a white light 50 is also equivalent to the spectrum of a red light 58, such as a red light emitting diode 12. Compare to broad spectra of the white light 52, the red light 58 is substantially monochromatic, being substantially devoid of colors in the green spectrum 59 and blue spectrum 60. Preferably, red spectrum 58 is defined as light with a wavelength of between about 570 nm and 730 nm.

FIGS. 2A and 2B disclose the spectral distribution of typical ophthalmic light sources. FIG. 2A represents a metal halide arc source and FIG. 2B represents a halogen filament source. As can be seen, both sources contain significant spectra below 570 nm, which could potentially be toxic to a patient's retina if used during posterior surgery.

FIG. 2C is a graphic illustration of a percent spectral reflection of a typical human retina. As can be seen, the mean reflectance of a retina in the preferred red light region is a relatively low 10-18 percent. Therefore, there is a significantly low risk of phototoxicity to a retina being illuminated by a red light source.

FIG. 3 is a schematic diagram of an ophthalmic illumination device 30 which is capable of producing red light 58 and white light 50. In this embodiment, a surgeon can alternatively select either an incandescent light 31 to produce white light 50 or a red LED 33 to produce red light 58. The process to select between the red light 58 and the white light 50 is essentially a switch 36 which could correspond with a physical switch of the illumination device 30 that is controlled by the surgeon. Illumination devices that produce a white source of light 50 are well known in the field of ophthalmic illumination as illustrated in FIGS. 2A and 2B, and it has also been known to use filters to remove ultraviolet 54 and infrared 56 radiation and thereby reduce the risk of phototoxicity. Additionally, various devices to direct the light into the eye 22 are well known in the field of ophthalmic surgery, such as fiber optic cable 35. A preferred feature of this invention is providing ophthalmic illumination device 30 with the capability to be switched between producing a white light 50 and a red light 58. Therefore, the use of the ophthalmic device 30 to illuminate an eye 22 is improved by adding the steps whereby a red spectrum 58, substantially devoid of a blue spectrum 60 and a green spectrum 59, is produced by the ophthalmic illumination device 30, and a surgeon selects between the red spectrum 58 and the white light 50 to illuminate an eye 22, thereby reducing the risk of phototoxicity.

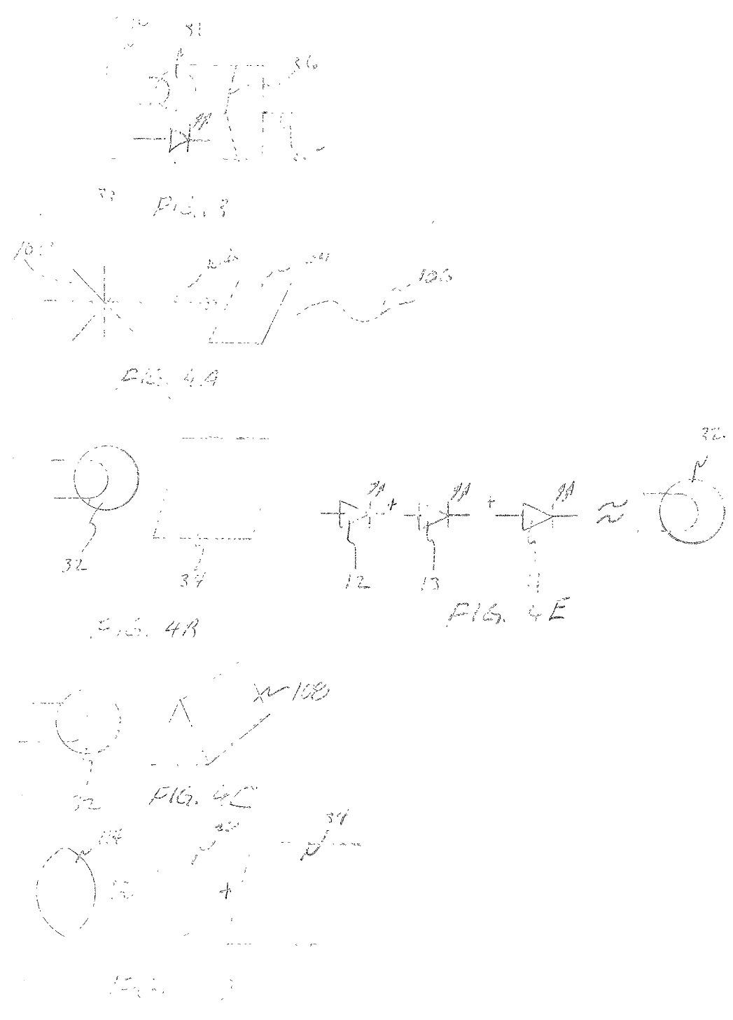

FIGS. 4A, 4B, and 4C illustrate several optical structures that may be substituted for the red light emitting diode (LED) 12 as equivalent features of the invention. This recitation of features will not limit a person skilled in the art from substituting other conventional and widely known optical devices that are a source of red light 58. FIG. 4A shows a red laser 100 in combination with a diffusion filter 104 producing incoherent, red light 106 in the same wavelength as the laser” red coherent light 102. FIG. 4B shows a source of white light 32 combined with a red filter 34 resting in the red spectrum 58. FIG. 4C shows white light source 32 passing light through a prism 108, where the white light is dissociated into a rainbow of the light's spectra; once the white light is dissociated through prism 108, the red spectrum 58 can be isolated and selectively passed. For example, a mirror (not shown) can intersect the red spectrum 58 and reflect it to the lens 20, or the prism 108 can be positioned to direct the red spectrum 58 directly onto the lens 20 that is coupled to the fiber optic cable 14.

It will be evident to those generally skilled in the art of illumination that combinations of certain other optical elements are equivalent to a single equivalent feature and would not depart from the scope of the invention. For example, the red LED 12 is equivalent to the source of white light 32 combined with the filter 34. Additionally, as shown in FIG. 4D, a red lens 114 could replace the red filter 34 and the lens 20 while simultaneously performing the same functions of both features. Similarly, as shown in FIG. 4E, the combination of a red LED 12, a blue LED 13, and a green LED 11 is functionally equivalent to a white light source 32.

FIG. 5 schematically illustrates an alternative means for directing the red light 58 into the eye 22 without the need of any incision 24. Red light 58 may be directed via LED 12 into an eye 22 through the pupil 26. A person skilled in the art of ophthalmic illumination will understand that conventional and widely known optical devices may be used to direct the light, including lenses and mirrors (not shown). For an ophthalmic microscope (not shown), it is likely that mirrors and lenses will be used to direct light through the pupil 26. For a vitreoretinal surgical machine (not shown), it is likely that a fiber optic cable 14 will be used to direct the light into the eye 22.

In view of the foregoing, it will be seen that the several advantages of the invention are achieved and attained. The embodiments were chosen and described in order to best explain the principles of the invention and its practical application to thereby enable others skilled in the art to best utilize the invention is various embodiments and with various modifications as are suited the particular use contemplated. As various modifications could be made in the constructions and methods herein described and illustrated without departing from the scope of the invention, it is intended that all matter contained in the foregoing description or shown in the accompanying drawings shall be interpreted as illustrative rather than limiting. For example, the lens 20 in FIG. 1 could be a lens with a graded index of refraction, also known as a GRIN lens and could 3extend through the housing module 15, coupling with the fiber optic cable 14 on the exterior side of the housing module 15; alternatively, the lens could be a combination of ordinary lenses that focuses the light onto the fiber optic cable. Thus, the breadth and scope of the present invention should be defined only in accordance with the following claims appended hereto and their equivalents.

What is claimed is:

1. An ophthalmic illumination device for reducing the risk of phototoxicity comprising:
a source of red light substantially devoid of any color in a blue spectrum or a green spectrum; and
a means for directing said red light into an eye.

2. An ophthalmic illumination device according to claim 1 wherein said source of red light comprises at least one red light emitting diode.

3. An ophthalmic illumination device according to claim 1 wherein said source of red light comprises at least one red laser having a diffusion filter.

4. An ophthalmic illumination device according to claim 1 wherein the wavelength of said red light is between about 570 nm and 730 nm.

5. An ophthalmic illumination device for reducing the risk of phototoxicity comprising:
a source of white light having a spectra of colors including a blue spectrum, a green spectrum, and a red spectrum;
a means for receiving said white light and primarily passing said red spectrum; and
a means for directing said red spectrum into an eye.

6. An ophthalmic illumination device according to claim 5 wherein said means for receiving said white light and primarily passing said red spectrum is a red filter.

7. An ophthalmic illumination device according to claim 5 wherein said means for receiving said white light and primarily passing said red spectrum is a prism.

8. An ophthalmic illumination device according to claim 5 wherein said source of white light is a red light emitting diode, a blue light emitting diode, and a green light emitting diode.

9. An ophthalmic illumination device for reducing the risk of phototoxicity comprising:
a source of white light having a spectra of colors including a blue spectrum, a green spectrum, and a red spectrum;
a means for receiving and passing said white light;
a means for receiving said white light and primarily passing said red spectrum;
a means for alternatively selecting between said white light and said red spectrum; and
a means for directing said elected light into an eye.

10. An ophthalmic illumination device for reducing the risk of phototoxicity comprising:
a source of white light having a spectra of colors including a blue spectrum, a green spectrum, and a red spectrum;
a source of red light substantially devoid of any color in said blue spectrum and said green spectrum;
a means for alternatively selecting between said red spectrum and said white light; and
a means for directing said selected light into an eye.

11. An ophthalmic illumination device according to claim 10 wherein said source of white light is a red light emitting diode, a blue light emitting diode, and a green light emitting diode.

12. An ophthalmic illumination device according to claim 11 wherein said source of red light is said red light emitting diode.

13. An ophthalmic illumination device according to claim 12 wherein said means for alternatively selecting between said red spectrum and said white light is a switch.

14. An ophthalmic illumination method for a surgeon to reduce the risk of phtotoxicity comprising the steps of:
producing a white light having a spectra of colors including a blue spectrum, a green spectrum, and a red spectrum;
producing a red light substantially devoid of any colors in said blue spectrum and said green spectrum;
selecting either said red light or said white light by the surgeon; and
directing said selected light into an eye.
